(19) United States
(12) Patent Application Publication
(10) Pub. No.: US 2022/0112526 A1
(43) Pub. Date: Apr. 14, 2022

(54) BIOSYNTHESIS OF VANILLIN FROM ISOEUGENOL

(71) Applicant: Conagen Inc., Bedford, MA (US)

(72) Inventors: Rui Zhou, Acton, MA (US); Junying Ma, Acton, MA (US); Oliver Yu, Lexington, MA (US)

(73) Assignee: Conagen Inc., Bedford, MA (US)

(21) Appl. No.: 17/514,050

(22) Filed: Oct. 29, 2021

Related U.S. Application Data
(63) Continuation of application No. PCT/US2020/030575, filed on Apr. 29, 2020.
(60) Provisional application No. 62/840,284, filed on Apr. 29, 2019.

(51) Int. Cl. C12P 7/24; C11B 9/00; A23L 33/105; C12N 9/02

(52) U.S. Cl. CPC C12P 7/24; C11B 9/0061; C12Y 113/11; C12N 9/0069; A23L 33/105

(57) ABSTRACT

The present invention relates to the production of vanillin via the bioconversion of isoeugenol. The bioconversion can be mediated in a cellular system (e.g., an Escherichia coli bacterium), or in an enzymatic reaction mixture without a cellular system.

### CROSS-REFERENCE TO RELATED APPLICATIONS

This application claims priority to U.S. Provisional Patent Application No. 62/840,284, filed on Apr. 29, 2019, the content of which is incorporated by reference herein in its entirety.

### FIELD OF THE INVENTION

The present disclosure generally relates to methods and materials for utilizing a fungal isoeugenol monooxygenase to catalyze the bioconversion of isoeugenol to vanillin in bacteria, yeasts or other cellular systems, or in non-cell systems.

### BACKGROUND OF THE INVENTION

Vanilla flavors are among some of the most frequently used flavors worldwide. They are used in the flavorings of numerous foods such as ice-cream, dairy products, desserts, confectionary, bakery products and spirits. They are also used in perfumes, pharmaceuticals and personal hygiene products.

Natural vanilla flavor has been obtained traditionally from the fermented pods of vanilla orchids. It is formed mainly after the harvest during several weeks of a drying and fermentation process of the beans by hydrolysis of vanillin glucoside that is present in the beans. The essential aromatic substance of vanilla flavor is vanillin (4-hydroxy 3-methoxybenzaldehyde).

Vanillin (4-hydroxy-3-methoxybenzaldehyde) is one of the most common flavor chemicals and is widely used in the food and beverage, perfume, pharmaceutical, and medical industries. About 12,000 tons of vanillin is consumed annually, of which only 20-50 tons are extracted from vanilla beans, the rest is produced synthetically, mostly from petrochemicals such as guaiacol and lignin. In recent years, increasing demands for natural flavors have led the flavor industry to produce vanillin by bioconversion, as the products of such bioconversion are considered natural by various regulatory and legislative authorities (e.g., European Community Legislation) when produced from biological sources such as living cells or their enzymes, and can be marketed as “natural products”.

Natural isoeugenol can be extracted from essential oils and is economical to use for the production of vanillin by enzymatic conversion or microbial bioconversion. Vanillin production via conversion of isoeugenol has been widely reported in a number of microorganisms, including Aspergillus niger, Bacillus subtilis, and Pseudomonas putida. However, the reported titers produced by these microorganisms were very low (less than 2 g/L), significantly limiting the practical application of this approach in the industry. Moreover, the reported bioconversion processes were complicated, further increasing the cost of vanillin production.

Accordingly, there is a need in the art for more cost-effective methods for producing vanillin with higher titers and conversion rates.

### SUMMARY OF THE INVENTION

The inventors have solved the above-mentioned problem by identifying a fungal isoeugenol monooxygenase which has a very low sequence identity with previously reported isoeugenol monooxygenases that have been used to produce vanillin. The identified fungal isoeugenol monooxygenase (CfIEM) showed surprisingly high activity for converting isoeugenol to vanillin. An expression plasmid harboring said CfIEM gene was constructed and an engineered host strain was developed and utilized to produce vanillin from isoeugenol at high titers and conversion rates. Recombinant proteins expressed by said CfIEM gene also can be isolated and purified and used to convert isoeugenol to vanillin in vitro.

Accordingly, in one aspect, the present disclosure relates to a bioconversion method of producing vanillin. The method can comprise expressing the CfIEM gene in a mixture, feeding isoeugenol to the mixture, and converting isoeugenol to vanillin. In some embodiments, the expressed CfIEM gene can have an amino acid sequence with at least 60% identity to SEQ ID NO.2, at least 65% identity to SEQ ID NO.2, at least 70% identity to SEQ ID NO.2, at least 75% identity to SEQ ID NO.2, at least 80% identity to SEQ ID NO.2, at least 85% identity to SEQ ID NO.2, at least 90% identity to SEQ ID NO.2, at least 95% identity to SEQ ID NO.2, or at least 99% identity to SEQ ID NO.2. In some embodiments, the expressed CfIEM gene can have an amino acid sequence identical to SEQ ID NO.2.

In various embodiments, the bioconversion method can include expressing the CfIEM gene by in vitro translation. In alternative embodiments, the bioconversion method can include expressing the CfIEM gene in a cellular system. In certain embodiments, the bioconversion method can include expressing the CfIEM gene in a bacterium or a yeast cell. The bioconversion method can include purifying the product from the step of expressing the CfIEM gene as a recombinant protein. In some embodiments, the purified recombinant protein can be added as a biocatalyst to a reaction mixture containing isoeugenol. In some embodiments, isoeugenol can be fed directly to the mixture in which the CfIEM gene is expressed.

The bioconversion method described herein can include recovering the vanillin from the mixture. The recovery of vanillin can be performed according to any conventional isolation or purification methodology known in the art. Prior to the recovery of the vanillin, the method also can include removing the biomass (enzymes, cell materials etc.) from the mixture.

In one aspect, the present disclosure relates to a method of producing vanillin using an isolated recombinant host cell, wherein the isolated recombinant host cell has been transformed with a nucleic acid construct that includes a polynucleotide sequence capable of encoding an isoeugenol monooxygenase. For example, the isoeugenol monooxygenase can have an amino acid sequence with at least 60% identity to SEQ ID NO.2, at least 65% identity to SEQ ID NO.2, at least 70% identity to SEQ ID NO.2, at least 75% identity to SEQ ID NO.2, at least 80% identity to SEQ ID NO.2, at least 85% identity to SEQ ID NO.2, at least 90% identity to SEQ ID NO.2, at least 95% identity to SEQ ID NO.2, or at least 99% identity to SEQ ID NO.2. In some embodiments, the isoeugenol monooxygenase can have an amino acid sequence identical to SEQ ID NO.2. In some embodiments, the method can include (i) cultivating the isolated recombinant host cell in a medium; (ii) adding isoeugenol to the medium to begin the bioconversion of isoeugenol to vanillin; and (iii) extracting vanillin from the medium. In other embodiments, the method can include (i) cultivating the isolated recombinant host cell in a medium to allow expression of the isoeugenol monooxygenase; (ii) isolating the isoeugenol monooxygenase; (iii) adding the isolated isoeugenol monooxygenase to a reaction mixture including isoeugenol; and (iv) extracting vanillin from the reaction medium.

In one aspect, the present disclosure relates to an isolated recombinant host cell transformed with a nucleic acid construct comprising a polynucleotide sequence encoding an isoeugenol monooxygenase, wherein the isoeugenol monooxygenase has an amino acid sequence with at least 60% identity to SEQ ID NO.2, at least 65% identity to SEQ ID NO.2, at least 70% identity to SEQ ID NO.2, at least 75% identity to SEQ ID NO.2, at least 80% identity to SEQ ID NO.2, at least 85% identity to SEQ ID NO.2, at least 90% identity to SEQ ID NO.2, at least 95% identity to SEQ ID NO.2, or at least 99% identity to SEQ ID NO.2. In some embodiments, the isoeugenol monooxygenase can have an amino acid sequence identical to SEQ ID NO.2. In some embodiments, the nucleic acid construct can contain a polynucleotide sequence that includes a sequence that is at least 70% identical to the nucleic acid sequence of SEQ ID NO. 1, 75% identical to the nucleic acid sequence of SEQ ID NO. 1, 80% identical to the nucleic acid sequence of SEQ ID NO. 1, 85% identical to the nucleic acid sequence of SEQ ID NO. 1, 90% identical to the nucleic acid sequence of SEQ ID NO. 1, or 95% identical to the nucleic acid sequence of SEQ ID NO. 1. In some embodiments, the nucleic acid construct can contain a polynucleotide sequence identical to SEQ ID NO. 1. In some embodiments, the isolated recombinant host cell can include a vector containing the isolated nucleic acid sequence of SEQ ID NO. 5. In various embodiments, the host cell can be selected from the group consisting of: a bacterium, a yeast, a filamentous fungus that is not Colletotrichum, a cyanobacterium, an alga, and a plant cell. For example, the host cell can be selected from the group of microbes consisting of Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveromyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; and Clostridium.

Vanillin produced using the methods and/or the isolated recombinant host cells described herein can be collected and incorporated into a consumable product. For example, the vanillin can be admixed with the consumable product. In some embodiments, the vanillin can be incorporated into the consumable product in an amount sufficient to impart, modify, boost or enhance a desirable taste, flavor, or sensation, or to conceal, modify, or minimize an undesirable taste, flavor or sensation, in the consumable product. The consumable product, for example, can be selected from the group consisting of food, food ingredients, food additives, beverages, drugs and tobacco. In some embodiments, the vanillin can be incorporated into the consumable product in an amount sufficient to impart, modify, boost or enhance a desirable scent or odor, or to conceal, modify, or minimize an undesirable scent or odor, in the consumable product. The consumable product, for example, can be selected from the group consisting of fragrances, cosmetics, toiletries, home and body care, detergents, repellents, fertilizers, air fresheners, and soaps.

Other features and advantages of the present invention will become apparent in the following detailed description, taken with reference to the accompanying drawings.

### BRIEF DESCRIPTION OF THE DRAWINGS

FIG. 1 shows the bioconversion pathway of isoeugenol to vanillin.

FIG. 2 provides a schematic diagram of the CfIEM-pET21a construct according to the present disclosure.

FIG. 3 provides a schematic diagram of the PpIEM-pET21a construct according to the present disclosure.

FIG. 4 provides a schematic diagram of the pUVAP plasmid according to the present disclosure.

FIG. 5 provides a schematic diagram of the CfIEM-pUVAP construct according to the present disclosure.

FIG. 6 provides a schematic diagram of the PpIEM-pUVAP construct according to the present disclosure.

FIG. 7 is a diagram showing the results of measurements of the purified recombinant proteins PpIEM (left) and CfIEM (right) by SDS-PAGE.

FIG. 8 provides HPLC chromatograms showing the bioconversion of isoeugenol to vanillin by CfIEM (middle panel) according to the present disclosure, compared to PpIEM (upper panel) and denatured CfIEM (lower panel).

FIG. 9 compares the production of vanillin by the E. coli strain IEUG-V02 (developed with CfIEM-pUVAP) according to the present disclosure against the E. coli strain IEUG-V02-control (developed with PpIEM-pUVAP).

FIG. 10 shows the production of vanillin with isoeugenol as the substrate by E. coli strain IEUG-V02 in a 5-liter fermenter according to the present disclosure.

### DETAILED DESCRIPTION

As used herein, the singular forms “a,” “an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean serving as an example, instance, or illustration. Any embodiment described herein as “exemplary” is not necessarily to be construed as preferred or advantageous over other embodiments.

“Cellular system” is any cells that provide for the expression of ectopic proteins. It included bacteria, yeast, plant cells and animal cells. It includes both prokaryotic and eukaryotic cells. It also includes the in vitro expression of proteins based on cellular components, such as ribosomes.

“Coding sequence” is to be given its ordinary and customary meaning to a person of ordinary skill in the art and is used without limitation to refer to a DNA sequence that encodes for a specific amino acid sequence.

“Growing” or “cultivating” a cellular system includes providing an appropriate medium that would allow cells to multiply and divide. It also includes providing resources so that cells or cellular components can translate and make recombinant proteins.

“Yeasts” are eukaryotic, single-celled microorganisms classified as members of the fungus kingdom. Yeasts are unicellular organisms which evolved from multicellular ancestors but with some species useful for the current invention being those that have the ability to develop multicellular characteristics by forming strings of connected budding cells known as pseudo hyphae or false hyphae.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing vanillin.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,’ and “peptide” are to be given their respective ordinary’ and customary meanings to a person of ordinary skill in the art; the three terms are sometimes used interchangeably and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term ‘polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art and is used without limitation to refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of ordinary skill in the art and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “transformed” or “recombinant”.

The terms “transformed,” “transgenic,” and “recombinant,” when used herein in connection with host cells, are to be given their respective ordinary and customary meanings to a person of ordinary skill in the art and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

“Protein Expression” refers to protein production that occurs after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the invention is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein. Polynucleotide molecules that have the activity genes of the current invention are capable of directing the production of vanillin and have a substantial percent sequence identity to the polynucleotide sequences provided herein and are encompassed within the scope of this invention.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

### Coding Nucleic Acid Sequences

The present invention relates to nucleic acid sequences that code for isoeugenol monooxygenase as described herein and which can be applied to perform the required genetic engineering manipulations. The present invention also relates to nucleic acids with a certain degree of “identity” to the sequences specifically disclosed herein. For example, aspects of the present invention encompass a nucleic acid sequence with at least 60% identity to SEQ ID NO. 1, at least 65% identity to SEQ ID NO. 1, at least 70% identity to SEQ ID NO. 1, at least 75% identity to SEQ ID NO. 1, at least 80% identity to SEQ ID NO. 1, at least 85% identity to SEQ ID NO. 1, at least 90% identity to SEQ ID NO. 1, at least 95% identity to SEQ ID NO. 1, or at least 99% identity to SEQ ID NO. 1. In some embodiments, the nucleic acid sequence used to encode an isoeugenol monooxygenase useful for the present invention can have a nucleic acid sequence identical to SEQ ID NO. 1.

The present invention also relates to nucleic acid sequences coding for an isoeugenol monooxygenase that has an amino acid sequence with at least 60% identity to SEQ ID NO.2, at least 65% identity to SEQ ID NO.2, at least 70% identity to SEQ ID NO.2, at least 75% identity to SEQ ID NO.2, at least 80% identity to SEQ ID NO.2, at least 85% identity to SEQ ID NO.2, at least 90% identity to SEQ ID NO.2, at least 95% identity to SEQ ID NO.2, or at least 99% identity to SEQ ID NO.2. In some embodiments, the isoeugenol monooxygenase can have an amino acid sequence identical to SEQ ID NO.2. In some embodiments, the present invention can relate to nucleic acid sequences coding for a functional equivalent of any of the foregoing.

### Constructs According to the Present Invention

In some aspects, the present invention relates to constructs like expression vectors for expressing an isoeugenol monooxygenase.

In an embodiment, the expression vector includes those genetic elements for expression of the recombinant polypeptide described herein (i.e., CfIEM) in various host cells. The elements for transcription and translation in the host cell can include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR). In molecular cloning, a vector is a DNA molecule used as a vehicle to artificially carry foreign genetic material into another cell, where it can be replicated and/or expressed (e.g. plasmid, cosmid, Lambda phages). A vector containing foreign DNA is considered recombinant DNA. The four major types of vectors are plasmids, viral vectors, cosmids, and artificial chromosomes. Of these, the most commonly used vectors are plasmids. Common to all engineered vectors are an origin of replication, a multicloning site, and a selectable marker.

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun et al, BIOTECHNIQUES 13, 515-18 (1992), each of which are incorporated herein by reference).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared using PCR appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into plant or microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector,

In some embodiments, the transformed cell is a plant cell, an algal cell, a fungal cell that is not Colletotrichum, or a yeast cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell expression systems and expression vectors containing regulatory sequences that direct high-level expression of foreign proteins that are well-known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Termination control regions may also be derived from various genes native to the microbial hosts. A termination site optionally may be included for the microbial hosts described herein.

Preferred host cells include those known to have the ability to produce vanillin from isoeugenol. For example, preferred host cells can include bacteria of the genus Escherichia and Pseudomonas.

### Fermentative Production of Vanillin

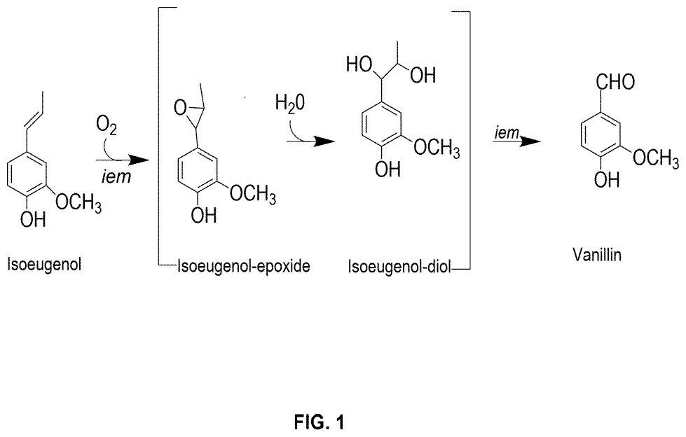

Isoeugenol is metabolized into vanillin through an epoxide-diol pathway involving oxidation of side chains of propenylbenzenes (FIG. 1). The inventors have surprisingly discovered that a putative isoeugenol monooxygenase from Colletotrichum fioriniae (CfIEM) showed surprisingly high activity in the bioconversion of isoeugenol to vanillin compared to previously reported bacterial IEMs. More specifically, by engineering a host strain that harbors the CfIEM gene and cultivating the engineered host strain in a mixture including isoeugenol, the inventors were able to achieve vanillin production at a titer above 10 g/L with a conversion rate of above 90%. The high titer and high conversion rate were obtained without the use of other crude enzymes and/or subfactors.

Cultivation of the host cells can be carried out in an aqueous medium in the presence of usual nutrient substances. A suitable culture medium, for example, can contain a carbon source, an organic or inorganic nitrogen source, inorganic salts and growth factors. For the culture medium, glucose can be a preferred carbon source. Yeast extract can be a useful source of nitrogen. Phosphates, growth factors and trace elements can be added.

The culture broth can be prepared and sterilized in a bioreactor. Engineered host strains according to the present invention can then be inoculated into the culture broth to initiate the growth phase. An appropriate duration of the growth phase can be about 5-40 hours, preferably about 10-35 hours and most preferably about 10-20 hours.

After the termination of the growth phase, the pH of the fermentation broth can be shifted to a pH of 8.0 or higher, and the substrate isoeugenol can be fed to the culture. A suitable amount of substrate-feed can be 0.1-40 g/L of fermentation broth, preferably about 0.3-30 g/L. The substrate can be fed as solid material or as aqueous solution or suspension. The total amount of substrate can be either fed in one step, in two or more feeding-steps, or continuously.

The bioconversion phase starts with the beginning of the substrate feed and lasts about 5-50 hours, preferably 10-40 hours, and most preferably 15-30 hours, namely until all substrate is converted to product and by-products.

After the terminated bioconversion phase, the biomass can be separated from the fermentation broth by any well-known method, such as centrifugation or membrane filtration and the like to obtain a cell-free fermentation broth.

An extractive phase can be added to the fermentation broth using, e.g., a water-immiscible—organic solvent, a plant oil or any solid extractant, e.g., a resin preferably, a neutral resin. The fermentation broth can be further sterilized or pasteurized. In some embodiments, the fermentation broth can be concentrated. From the fermentation broth, vanillin can be extracted selectively using, for example, a continuous liquid-liquid extraction process, or a batch-wise extraction process.

Advantages of the present invention include, among others, the ability to perform both the growth phase and the subsequent bioconversion phase in the same medium. This highly simplifies the production process, making the process efficient and economical, thus allowing scale-up to industrial production levels.

One skilled in the art will recognize that the vanillin composition produced by the method described herein can be further purified and mixed with fragrant and/or flavored consumable products as described above, as well as with dietary supplements, medical compositions, cosmeceuticals, for nutrition, as well as in pharmaceutical products.

The disclosure will be more fully understood upon consideration of the following non-limiting Examples. It should be understood that these examples, while indicating preferred embodiments of the subject technology, are given by way of illustration only. From the above discussion and these examples, one skilled in the art can ascertain the essential characteristics of the subject technology, and without departing from the spirit and scope thereof, can make various changes and modifications of the subject technology to adapt it to various uses and conditions.

### EXAMPLES

### Bacterial Strains, Plasmids and Culture Conditions.

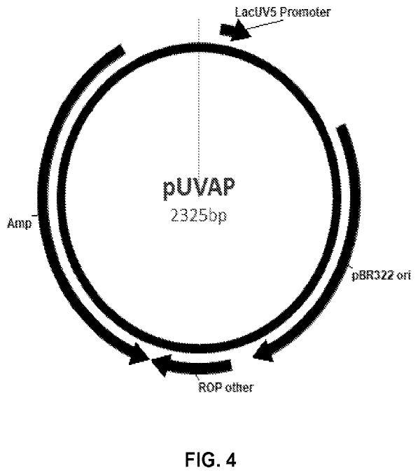

E. coli strains of DH5a and BL21 (DE3) were purchased from Invitrogen. E. coli strain W3110 was obtained from the Coli Genetic Stock Center, E. coli Genetic Resources at Yale University (http://cgsc2.biology.yale.edu/). Plasmid pET21a was purchased from EMD Millipore (Billerica, Mass., USA). Plasmid pUVAP was constructed by the inventors with the nucleotide sequence listed in SEQ ID NO: 5 and the map shown in FIG. 4, which was used for both gene cloning and gene expression purposes.

### DNA Manipulation.

All DNA manipulations were performed according to standard procedures. Restriction enzymes, T4 DNA ligase were purchased from New England Biolabs. All PCR reactions were performed with New England Biolabs' Phusion PCR system according to the manufacturer's guidance.

### Example 1: Identification of Target Genes

A DNA fragment with a full ORF (open reading frame) of 1431 bp, namely CfIEM from the fungus Colletotrichum fioriniae PJ7, was identified from NCBI database (EXF85749.1). It was annotated to be an isoeugenol monooxygenase. The ORF encodes a protein with 539 amino acids (SEQ ID NO: 2), a theoretical molecular weight of 60.80 kDa and a calculated isoelectric point (pI) of 5.47. The corresponding nucleotide was synthesized by GeneUniversal Inc. Company (Newark, N.J., USA) after codon optimization for expression in Escherichia coli (SEQ ID NO: 1).

PpIEM, an isoeugenol monooxygenase from Pseudomonas putida, GenBank Accession ID No: AB291707.1, has been functional characterized and previously used for vanillin production. See Yamada et al., “Biotransformation of Isoeugenol to Vanillin by Pseudomonas putida 1E27 cells,” Appl. Microbiol. Biotechnol., 73(5): 1025-1030 (2007), and Yamada et al., “Vanillin production using Escherichia coli cells over-expressing isoeugenol monooxygenase of Pseudomonas putida,” Biotechnol. Lett., 30:665-670 (2008). In order to compare the activity of CfIEM with PpIEM in converting isoeugenol to vanillin, PpIEM was synthesized by GeneUniversal Inc Company (Newark, N.J., USA), using the nucleotide sequence listed as SEQ ID NO: 3. The corresponding amino acid sequence is listed as SEQ ID NO: 4.

Bioinformatic analysis indicates CfIEM shows low identity with isoeugenol monooxygenases reported from Pseudomonas species. Specifically, bioinformatic analysis indicates that CfIEM only has 34% and 35% identity with PpIEM, IEM from Pseudomonas putida, and PnIEM, an IEM from Pseudomonas nitroreducens, respectively.

### Example 2: Construction of Plasmids

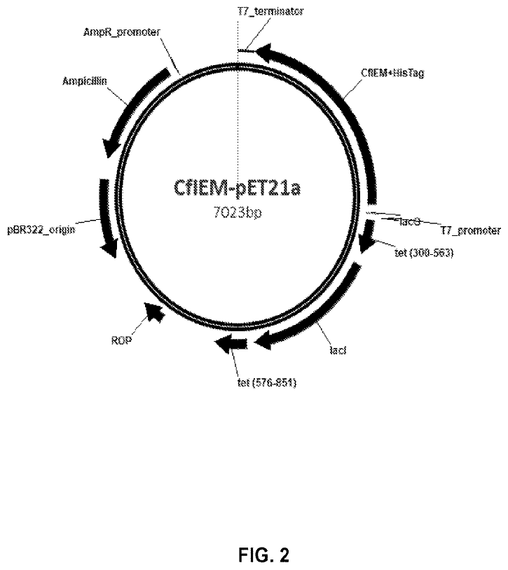

The open reading frames (ORF) of CfIEM was cloned into the Nde I/Not I restriction sites of pET21a with the removal of stop codon so that the recombinant protein has a 6×His tag at the C-terminal. The ORF of CfIEM was amplified with an introduction of Nde I restriction site at the 5′-end and that of Not I site at the 3′-end with a pair of primers of CfIEM-NdeI-F and CfIEM-NotI-R1 in Table 1. After digested with Nde I and Not I, the PCR fragment was ligated into the restriction sites of Nde I and Not I of expression vector pET21a, and transformed into DH5alpha competent cells, generating a plasmid of CfIEM-pET21a (FIG. 2). After sequencing confirmation, the plasmid was ready for transformation into Escherichia coli strain BL21 (DE3).

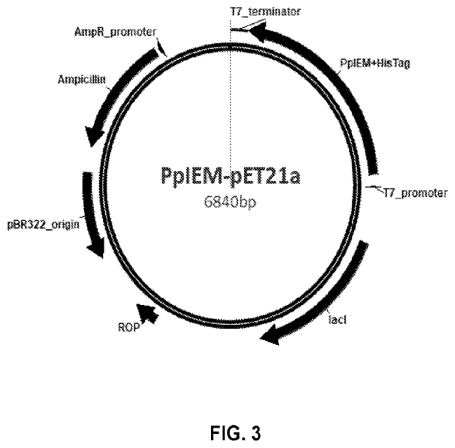

As a control, PpIEM was cloned into the Nde I/Not I restriction sites of pET21a with the removal of stop codon so that the recombinant protein has a 6×His tag at the C-terminal with the similar protocol as above. The ORF of PpIEM was amplified with an introduction of Nde I restriction site at the 5′-end and that of Not I site at the 3′-end with a pair of primers of PpIEM-NdeI-F and PpIEM-NotI-R1 in Table 1. After digested with Nde I and Not I, the PCR fragment was ligated into the restriction sites of Nde I and Not I of expression vector pET21a, and transformed into DH5alpha competent cells, generating a plasmid of PpIEM-pET21a (FIG. 3). After sequencing confirmation, the plasmid was ready for transformation into Escherichia coli strain BL21 (DE3).

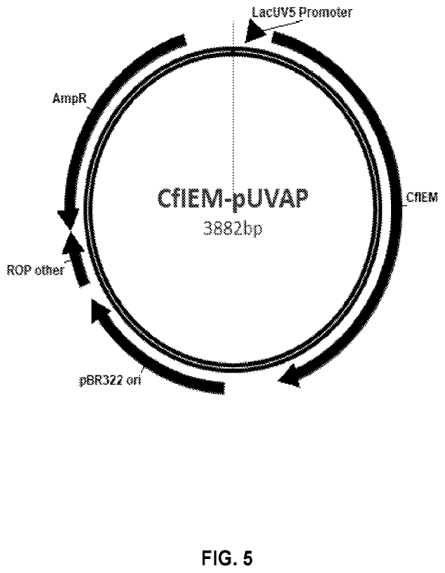

For generating Escherichia coli strains for the bioconversion of isoeugenol to vanillin in fermenter, the open reading frame of CfIEM was cloned into Nde I/Not I site of pUVAP vector (FIG. 4) to generate an expression vector of CfIEM-pUVAP (FIG. 5) with the same above procedure except the primers used were CfIEM-Nde-F and CfIEM-NotI-R2. The expressed CfIEM with this vector has no His-Tag.

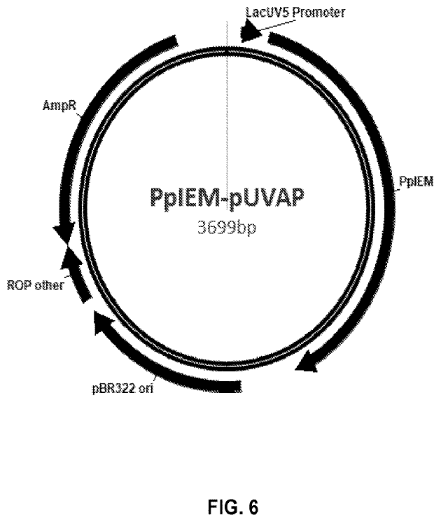

As the control, PpIEM was cloned into Nde I/Not I site of pUVAP vector to generate an expression vector of PpIEM-pUVAP (FIG. 6) with the same procedures described above except the primers used were PpIEM-Nde-F and PpIEM-NotI-R2. The expressed PpIEM with this vector has no His-Tag.

TABLE 1
Primers for construction of CfIEM and
PpIEM expression vectors

| | SEQ | |
|---|---|---|
| Primer | ID | |
| name | NO: | Nucleotide Sequence |
| CfIEM- | 6 | GGAATTCCATATGGCACCGGGTCGTACCGATGATGA |
| NdeI-F | | AGC |
| CfIEM- | 7 | AAGGAAAAAAGCGGCCGCCAGCGGTTCCAGTGCACC |
| NotI- | | CTGACC |
| R1 | | |
| CfIEM- | 8 | AAGGAAAAAAGCGGCCGCTTACAGCGGTTCCAGTGC |
| NotI- | | ACCCTGACC |
| R2 | | |
| PpIEM- | 9 | GGAATTCCATATGAGCGCACCGGAAGAACATCATGG |
| NdeI-F | | |
| PpIEM- | 10 | AAGGAAAAAAGCGGCCGCTGCCAGCGGTTCCAGTGC |
| NotI- | | ACCACG |
| R1 | | |
| PpIEM- | 11 | AAGGAAAAAAGCGGCCGCTTATGCCAGCGGTTCCAG |
| NotI- | | TGCACCACG |
| R2 | | |

### Example 3: Transformation of E. coli Cells with the Developed Constructs

The construct of CfIEM-pET21a was introduced into E. coli BL21 (DE3) cells with standard chemical transformation protocol to generate a strain of ISEG-V01. With the same protocol, a strain of ISEG-V01-control was developed with PpIEM-pET21a. The strains of ISEG-V01 and ISEG-V01-control were used for expression of recombinant proteins and functional characterization of the recombinant proteins.

The plasmid of CfIEM-pUVAP was introduced into E. coli W3110 competent cells with standard chemical transformation protocol to generate a strain of IEUG-V02. A control strain of IEUG-V02-control was developed by introducing the plasmid of PpIEM-pUVAP into E. coli W3110 competent cells using the same procedures. The strains IEUG-V02 and IEUG-02-control were used for bioconversion of isoeugenol to vanillin with the whole cells.

### Example 4: Heterologous Expression of CfIEM in E. coli and Purification of the Recombinant Protein

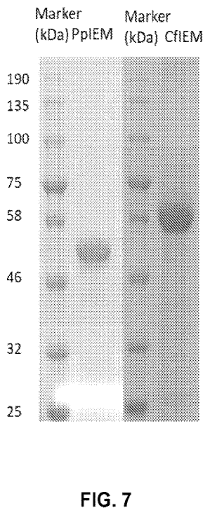

A single colony of E. coli strain ISEG-V01 was grown in 5 mL of LB medium with 100 mg/L of ampicillin at 37° C. overnight. This seed culture was transferred to 200 mL of LB medium with 100 mg/L of ampicillin. The cells were grown at 37 C at 250 rpm to OD600 of 0.6-0.8, and then IPTG was added to a final concentration of 0.5 mM and the growth temperature was changed to 16° C. The E. coli cells were harvested after 16 hours of IPTG induction by centrifugation at 4000 g for 15 min at 4° C. The resultant pellet was re-suspended in 5 mL of 100 mM Tris-HCl, pH 7.4, 100 mM NaOH, 10% glycerol (v/v), and sonicated for 2 min on ice. The mixture was centrifuged at 4000 g for 20 min at 4° C. The recombinant protein CfIEM in the supernatant was purified with His60 Ni Superflow resin from Clonetech Inc. following the manufacturer's protocol. The same procedures were used to grow a single colony of E. coli strain ISEG-V01-control to obtain the recombinant protein PpIEM. The purification of the two recombinant proteins was checked by SDS-PAGE (FIG. 7).

### Example 5: Enzyme Assay In Vitro

The two purified isoeugenol monooxygenases were assayed by measuring the formation of vanillin in an enzymatic measuring mixture with isoeugenol as the substrate. The reaction mixture contained 10 mM isoeugenol, 100 mM potassium phosphate buffer (pH 7.0), 10% (v/v) ethanol and an appropriate amount of enzyme, in a total volume of 1 ml. The reaction was started, by adding isoeugenol as ethanol solution, carried out at 30° C. for 10 min with reciprocal shaking (160 strokes min⁻¹) and stopped with 1 ml of methanol. After centrifugation at 21,500, the supernatant was analyzed by HPLC for the determination of isoeugenol, vanillin, and vanillyl alcohol. CfIEM enzyme treated in boiling water for 5 minutes was used as the negative control.

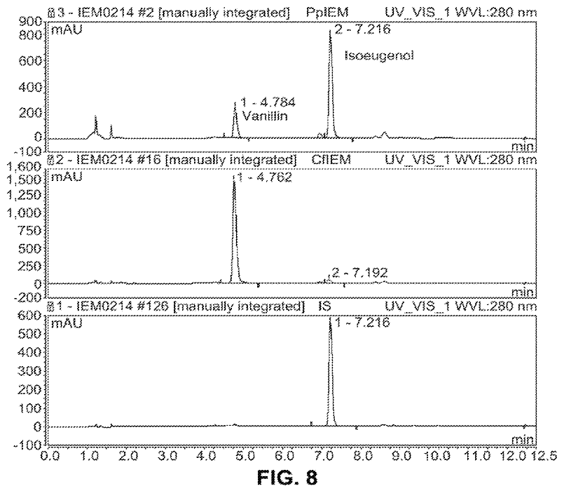

FIG. 8 shows HPLC chromatograms obtained with the supernatants from the experiments using purified PpIEM (upper panel), purified CfIEM (middle panel), and the negative control denatured CfIEM (lower panel). As shown, purified CfIEM showed high activity toward isoeugenol. Almost all isoeugenol added to the reaction mixture was converted to vanillin (middle panel). By comparison, the purified PpIEM only converted a small portion of added isoeugenol to vanillin (upper panel). As expected, no vanillin was produced with the negative control (lower panel).

### Example 6: Bioconversion of Isoeugenol to Vanillin In Vivo with Shaking Flasks

E. coli W3110 strains of IEUG-V02 and IEUG-V02-control were grown in LB medium with 50n/L ampicillin in 3 ml LB at 37° C. overnight as the seed cultures. The seed culture of 0.2 mL was inoculated into 20 mL M9 medium with an inclusion of 5 g/L of yeast extract and 50n/L ampicillin in 125 mL of shaking flasks. The cells were grown in a shaker with shaking speed of 250 rpm for 6 hours at 30° C., and then 400 μl 20% isoeugenol (v/v in DMSO) was added into the flasks. Two more time of 200 μl 20% isoeugenol (v/v in DMSO) addition into the culture was performed after 12 and 24 hours of the first-time addition. The culture mixture of 100 μl was taken from the flasks at indicated time interval respectively, and mixed well with 900 μl of methanol by vortex and then centrifuged at 20,000 g for 15 min. The resultant supernatant of 50 uL was taken into a HPLC sample vial for HPLC analysis. The experiment was performed in triplicates.

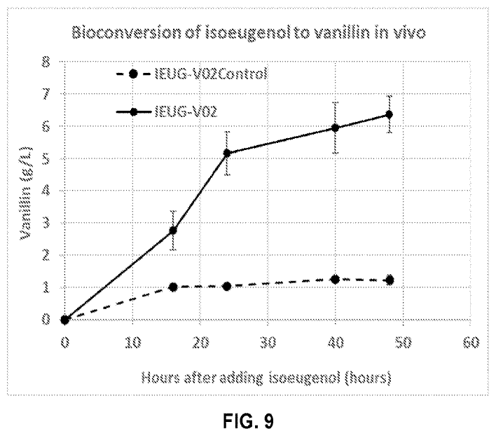

FIG. 9 compares the production of vanillin by the E. coli strain IEUG-V02 (developed with CfIEM-pUVAP) against the E. coli strain IEUG-V02-control (developed with PpIEM-pUVAP). As shown in FIG. 9, both strains harboring CfIEM and PpIEM, respectively, converted isoeugenol to vanillin in the culture medium. However, the conversion rate of IEUG-V02 harboring CfIEM was significantly higher than that of the strain harboring PpIEM. The strain harboring PpIEM (IEUG-V02-control) produced only about 1 g/L of vanillin almost 50 hours after the addition of isoeugenol. By comparison, the strain harboring CfIEM (IEUG-V02) produced over 6 g/L of vanillin at the end of the bioconversion period under the same conditions.

### Example 7: Bioconversion of Isoeugenol to Vanillin in Fermenters

A fermentation process was developed to use IEUG-V02 strain to produce natural vanillin with natural isoeugenol as the substrate in fermenters. Specifically, glycerol stock of ISEG-V02 of 1 mL was inoculated into 100 mL seed culture medium (Luria-Bertani medium with 5 g/L yeast extract, 10 g/L tryptone, 10 g/L NaCl, and 50 mg/L ampicillin) in 500 mL flasks. The seed was cultivated in a shaker with shaking speed of 200 rpm at 37° C. for 8 hours, and then transferred into 3 liter of fermentation medium of Luria-Bertani medium plus 10 g/L initial glucose, 50 mg/L ampicillin in a 5-liter fermenter.

The 5-liter vanillin fermenter process has two phases, cell growth phase and bioconversion phase. Cell growth phase was from elapsed fermentation time (EFT) 0 hour to about EFT 16.5 hours. The fermentation parameters were set as follows: Air flow: 0.6 vvm; pH was not below 7.1 controlled by using 4N NaOH. The growth temperature was set to 30° C. and the agitation was set to 300-500 rpm. The dissolved oxygen (DO) was cascaded to agitation to maintain above 30%. The growth time was about 16-17 hours.

Bioconversion phase was from EFT 16.5 hour to 46.5 hour. The fermentation parameters were set as follows: Air flow: 0.4 vvm. pH was controlled to not below 8.0 with 4N NaOH, and the temperature was 30° C. Agitation was set to 250-500 rpm and DO was maintained above 30% by cascaded to agitation. Isoeugenol was fed at a feeding rate of 0.6 g/L at EFT 16.5 hour, and then the rate was reduced to 0.4 g/L at EFT 27.5 hour until EFT 44 hour and the fermentation phase was completed at EFT 46-47 hour.

The culture mixture was taken from the fermenter at indicated time intervals. HPLC analyses of isoeugenol, vanillin, and vanillyl alcohol were carried out with a Vanquish Ultimate 3000 system. Intermediates were separated by reverse-phase chromatography on a Dionex Acclaim 120 C18 column (particle size 3 μm; 150 by 2.1 mm) with a gradient of 0.2% (volume/volume) trifluoric acid (eluant A) and acetonitrile with 0.2% (volume/volume) (eluant B) in a range of 10 to 40% (vol/vol) eluant B and at a flow rate of 0.6 ml/min. For quantification, all intermediates were calibrated with external standards. The compounds were identified by their retention times, as well as the corresponding spectra, which were identified with a diode array detector in the system.

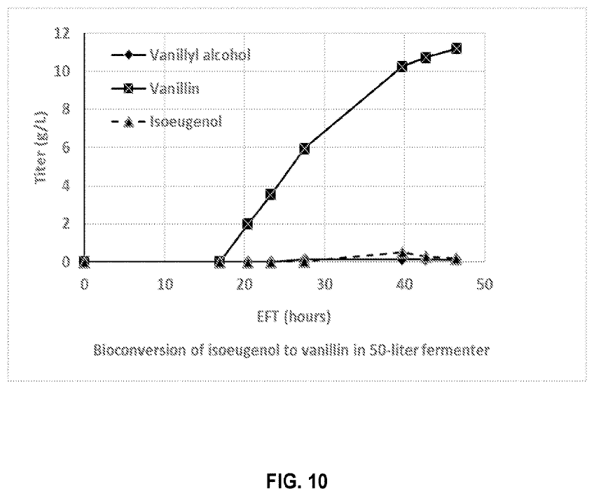

FIG. 10 shows the amounts of vanillin produced, against the amounts of isoeugenol and vanillyl alcohol produced, over the bioconversion phase from EFT 16.5 hour to 46.5 hour. As shown in FIG. 10, the vanillin production titer reached above 10 g/L with a conversion rate from isoeugenol of above 90%.

As is evident from the foregoing description, certain aspects of the present disclosure are not limited by the particular details of the examples provided herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

Sequences of Interest
SEQ ID NO. 1: Nucleic Acid Sequence of CfIEM
ATGGCACCGGGTCGTACCGATGATGAAGCAGCAGAAGTTGCAACCAGCAG
CAAAACCCATGCAATTAGCAATTATGGTCTGACCACCAAATGGCCTGAAG
CATACGATCTGGCAGGTAGCAATCTGCCGTGTCGTCTGGAAGGTGAAATT
GGTGATCTGGTTGTTCTGGGTGAAATTCCGAAAGAAATTGATGGCACCTT
TTATCGTGTTATGACCGATCCGTTTGTTCCGCCTCATCCGAATAATGTTC
CGCTGGATGGTGATGGTAATATTAGCGCATTTCGCATTAAAGATGGTCGC
GTGGATATGAAAATGCGTTATGTTGAAACCGAGCGCTATAAACTGGAACG
CAAAGCAAATAAAGCACTGTTTGGTCTGTATCGCAATCCGTTTACACATC
ATCCGTGTGTTCGTGCAGCAGTTGATAGCACCGCAAATACCAATGTTGTT
CTGTGGGCAGATCATTTTCTGGCACTGAAAGAAGGTGGTCTGCCGTATAG
CGTTGATCCGCAGACACTGGAAACCATCAAATATGATCCTTTTGGCCAGA
TCAAAGCCAAAACCTTTACCGCACATCCGAAAATTGATCCGTATACCAAT
GAACTGGTGGTGTTTGGTTATGAAGCACGTGGTCTGGCAAGCCTGGATAT
TGTTATTTATGCACTGGATAGCAACGGCGTGAAACATGATGAACAGTGGA
TTAAAAGCCCGTGGTGTGCACCGATTCATGATTGTGCAATTACCCCGAAT
TGGATTATTCTGGCCCTGTGGCCGTTTGAAGCAAGCGTGGATCGTATGAA
AAAAGGCGGTCATCATTGGGCATGGGATTATGATTTACCGGCAACCTTTA
TTGTTGCACCGCGTCGTAGCAGCACCAAACTGCCTCCAGGTTGGCAGCCT
GGTGAAAGCCGTGTTTATAGCTGGAAAAATTGCATGCTGATTCATACCGG
TGGTGCATGGGAAAGCGAAGATGGTCAGACACTGTTTATGGAAACCACAC
GTGTTCATGATAATGGCTTTCCGTTTTTTCCGACCAATGAAGGTGTTTAT
CCGGCACCTGATCCGAAAAGCGATTATGTTCGTTGGACATTTGATCTGAG
TCAGCCGACCAATAGCAAAGTTCCTGATCCGGAAATTATCCTGGATATGC
CTGCAGAATTTCCGCGTCTGGATGAACGTTTTCTGAGCAAAAGCTATAAT
TATGTGTGGATCAACGTGTACATCCACGAAGATCAGAACGGTGATGGTCA
TACCATTGTTGAAGGTCTGAATGGTCTGGCCATGCATAATAGCAAAACCA
AAGAAACCAAATGCTTTAATGCCGGTGCCGAAAGTTTTTGTCAAGAACCG
ATCTTTATTCCGCGTAGTGATGATGCACCGGAAGGTGATGGTTGGGTTAT
GACAATGGTTGAACGTCGTGGTGCAAATCGTAATGAAATTGTTGTGCTGG
ATACCAAGGATTTCGAAAAACCGATTGCAGTTATTCAGCTGCCGTTTCAT
GTTAAAGCACAGGTTCATGGTAATTGGGTTGATCAGCGTCGTCTGAAAGC
ATGGCAGAAACTGGCACGTGATGCAGTGCCGGAACCGATTAGCGGTCAGG
GTGCACTGGAACCGCTGTAA
SEQ ID NO. 2 Amino Acid Sequence of CfIEM
MAPGRTDDEAAEVATSSKTHAISNYGLTTKWPEAYDLAGSNLPCRLEGEI
GDLVVLGEIPKElDGTFYRVMTDPFVPPHPNNVPLDGDGNISAFRIKDGR
VDMKMRYVETERYKLERKANKALFGLYRNPFTHHPCVRAAVDSTANTNVV
LWADHFLALKEGGLPYSVDPQTLETIKYDPFGQIKAKTFTAHPKIDPYTN
ELVVFGYEARGLASLDIVIYALDSNGVKHDEQWIKSPWCAPIHDCAITPN
WIILALWPFEASVDRMKKGGHHWAWDYDLPATFIVAPRRSSTKLPPGWQP
GESRVYSWKNCMLIHTGGAWESEDGQTLFMETTRVHDNGFPFFPTNEGVY
PAPDPKSDYVRWTFDLSQPTNSKVPDPEIILDMPAEFPRLDERFLSKSYN
YVWINVYIHEDQNGDGHTIVEGLNGLAMHNSKTKETKCFNAGAESFCQEP
IFIPRSDDAPEGDGWVMTMVERRGANRNEIVVLDTKDFEKPIAVIQLPFH
VKAQVHGNWVDQRRLKAWQKLARDAVPEPISGQGALEPL
SEQ ID NO. 3 Nucleic Acid Sequence of PpIEM
ATGGCGAGACTCAACCGCAACGACCCGCAATTAGTAGGAACACTTCTCCC
CACCCGTATCGAGGCAGACTTGTTCGATCTAGAGGTTGACGGCGAAATCC
CAAAATCAATAAATGGAACGTTCTACCGTAATACGCCAGAACCTCAAGTT
ACCCCGCAAAAATTCCACACCTTCATAGATGGAGATGGAATGGCCTCTGC
CTTCCACTTCGAAGATGGTCATGTCGACTTCATCAGTCGCTGGGTTAAAA
CCGCTCGATTCACGGCCGAACGACTAGCGCGAAAATCGCTATTTGGCATG
TACAGAAACCCCTATACCGACGACACCAGTGTAAAAGGACTAGACCGCAC
CGTTGCCAATACAAGCATCATTAGCCATCACGGCAAGGTGCTGGCGGTGA
AGGAAGACGGCCTACCGTACGAACTGGATCCTCGTACACTTGAAACTCGC
GGACGCTTCGACTACGACGGCCAAGTTACCAGCCAAACCCACACCGCCCA
TCCAAAATATGACCCGGAAACGGGTGACTTGTTGTTCTTCGGTTCGGCAG
CTAAGGGCGAAGCAACTCCAGACATGGCCTATTACATTGTCGACAAGCAC
GGCAAGGTGACACATGAAACTTGGTTTGAGCAGCCCTATGGCGCATTCAT
GCACGACTTTGCCATTACCCGAAATTGGTCCATTTTCCCAATTATGCCGG
CCACCAACAGCCTGTCCCGCCTCAAGGCGAAACAGCCAATTTATATGTGG
GAGCCGGAACTGGGCAGCTACATTGGCGTACTGCCGCGCCGCGGCCAGGG
CAGTCAGATTCGCTGGCTCAAGGCACCGGCGCTCTGGGTATTTCATGTTG
TGAATGCTTGGGAAGTCGGAACCAAGATTTATATCGACCTTATGGAAAGT
GAAATCCTGCCGTTCCCCTTCCCCAACTCACAAAACCAACCCTTCGCCCC
TGAGAAAGCCGTACCACGCCTGACTCGTTGGGAAATTGACCTCGATAGCA
GCAGCGACGAGATCAAGCGAACCCGGCTACACGATTTCTTTGCGGAAATG
CCAATCATGGATTTTCGCTTCGCCCTGCAATGCAACCGCTATGGCTTTAT
GGGGGTGGACGATCCACGCAAACCACTTGCGCATCAGCAGGCCGAGAAGA
TATTTGCGTACAACTCACTCGGCATCTGGGACAACCACCGAGGTGACTAC
GACCTCTGGTACTCCGGAGAAGCCTCGGCGGCCCAGGAGCCGGCCTTCGT
CCCTAGAAGTCCGACCGCCGCCGAAGGTGATGGGTACTTGCTGACCGTGG
TTGGTCGTCTCGATGAAAATCGCAGTGATCTGGTAATTCTCGACACTCAA
GACATCCAGTCTGGTCCCGTGGCAACCATCAAGCTGCCATTCCGGTTAAG
GGCCGCTCTCCATGGCTGCTGGGTACCCAGACCTTAA
SEQ ID NO. 4 Amino Acid Sequence of PpIEM
MATFDRNDPQLAGTMFPTRIEANVFDLEIEGEIPRAINGSFFRNTPEPQV
TTQPFHTFIDGDGLASAFHFEDGQVDFVSRWVCTPRFEAERSARKSLFGM
YRNPFTDDPSVEGIDRTVANTSIITHHGKVLAAKEDGLPYELDPQTLETR
GRYDYKGQVTSHTHTAHPKFDPQTGEMLLFGSAAKGERTLDMAYYIVDRY
GKVTHETWFKQPYGAFMHDFAVTRNWSIFPIMPATNSLERLKAKQPIYMW
EPERGSYIGVLPRRGQGKDIRWFRAPALWVFHVVNAWEEGNRILIDLMES
EILPFPFPNSQNLPFDPSKAVPRLTRWElDLNSGNDEMKRTQLHEYFAEM
PIMDFRFALQDHRYAYMGVDDPRRPLAHQQAEKIFAYNSLGVWDNHRKDY
ELWFTGKMSAAQEPAFVPRSPDAPEGDGYLLSVVGRLDEDRSDLVILDTQ
CLAAGPVATVKLPFRLRAALHGCWQSKN
SEQ ID NO. 5 Nucleic Acid Sequence of
Plasmid pUVAP
GGCAGTGAGCGCAACGCAATTAATGTGAGTTAGCTCACTCATTAGGCACC
ATCGTTTAGGCACCCCAGGCTTTACACTTTATGCTTCCGGCTCGTATAAT
GTGTGGAATTGTGAGCGGATAACAATTTCAACTATAAGAAGGAGATATAC
ATATGGCGGATCCGAATTCGGCGCGCCAGATCTCAATTGGATATCGGCCG
GCCGACGTCGGTACCGCGGCCGCCACCGCTGAGCAATAACTAGCATAACC
CCTTGGGGCCTCTAAACGGGTCTTGAGGGGTTTTTTGCTGAAAGGAGGAA
CTATATCCGGGTAACGAATTCAAGCTTGATATCATTCAGGACGAGCCTCA
GACTCCAGCGTAACTGGACTGCAATCAACTCACTGGCTCACCTTCACGGG
TGGGCCTTTCTTCGGTAGAAAATCAAAGGATCTTCTTGAGATCCTTTTTT
TCTGCGCGTAATCTGCTGCTTGCAAACAAAAAAACCACCGCTACCAGCGG
TGGTTTGTTTGCCGGATCAAGAGCTACCAACTCTTTTTCCGAGGTAACTG
GCTTCAGCAGAGCGCAGATACCAAATACTGTTCTTCTAGTGTAGCCGTAG
TTAGGCCACCACTTCAAGAACTCTGTAGCACCGCCTACATACCTCGCTCT
GCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGC
TGAACGGGGGGTTCGTGCACACAGCCCAGCTTGGAGCGAACGACCTACAC
CGAACTGAGATACCTACAGCGTGAGCTATGAGAAAGCGCCACGCTTCCCG
AAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCC
TGTCGGGTTTCGCCACCTCTGACTTGAGCATCGATTTTTGTGATGCTCGT
CAGGGGGGCGGAGCCTATGGAAAAACGCCAGCAACGCAGAAAGGCCCACC
CGAAGGTGAGCCAGGTGATTACATTTGGGCCCTCATCAGAGGTTTTCACC
GTCATCACCGAAACGCGCGAGGCAGCTGCGGTAAAGCTCATCAGCGTGGT
CGTGAAGCGATTCACAGATGTCTGCCTGTTCATCCGCGTCCAGCTCGTTG
AGTTTCTCCAGAAGCGTTAATGTCTGGCTTCTGATAAAGCGGGCCATGTT
AAGGGCGGTTTTTTCCTGTTTGGTCATTTACCAATGCTTAATCAGTGAGG
CACCTATCTCAGCGATCTGTCTATTTCGTTCATCCATAGTTGCCTGACTC
CCCGTCGTGTAGATAACTACGATACGGGAGGGCTTACCATCTGGCCCCAG
TGCTGCAATGATACCGCGAGACCCACGCTCACCGGCTCCAGATTTATCAG
CAATAAACCAGCCAGCCGGAAGGGCCGAGCGCAGAAGTGGTCCTGCAACT
TTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCA
TCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGT
TATCACTCATGGTTATGGCAGCACTGCATAATTCTCTTACTGTCATGCCA
TCCGTAAGATGCTTTTCTGTGACTGGTGAGTACTCAACCAAGTCATTCTG
AGAATAGTGTATGCGGCGACCGAGTTGCTCTTGCCCGGCGTCAATACGGG
ATAATACCGCGCCACATAGCAGAACTTTAAAAGTGCTCATCATTGGAAAA
CGTTCTTCGGGGCGAAAACTCTCAAGGATCTTACCGCTGTTGAGATCCAG
TTCGATGTAACCCACTCGTGCACCCAACTGATCTTCAGCATCTTTTACTT
TCACCAGCGTTTCTGGGTGAGCAAAAACAGGAAGGCAAAATGCCGCAAAA
AAGGGAATAAGGGCGACACGGAAATGTTGAATACTCATAGCTCCTGAAAA
TCTCGATAACTCAAAAAATACGCCCGGTAGTGATCTTATTTCATTATGGT
GAAAGTTGGAACCTCTTACGTGCCGATCAAGTCAAAAGCCTCCGGTCGGA
GGCTTTTGACTTTCTGCTATGGAGGTCAGGTATGATTTAAATGGTCAGTA
TTGAGCGATATCTAGAGAATTCGTC

1. A bioconversion method of producing vanillin comprising:
a. expressing a CfIEM gene in a mixture, wherein the expressed CfIEM gene has an amino acid sequence with at least 70% identity to SEQ ID NO. 2 in the mixture;
b. feeding isoeugenol to the mixture; and
c. converting isoeugenol to vanillin.

2. (canceled)

3. The method of claim 1, wherein the expressed CfIEM gene has an amino acid sequence with at least 90% identity to SEQ ID NO. 2.

4. The method of claim 1, wherein the expressed CfIEM gene has an amino acid sequence with at least 95% identity to SEQ ID NO. 2.

5. The method of claim 1, wherein the step of expressing the CfIEM gene is selected from the group consisting of: expressing the gene by in vitro translation; expressing the gene in a cellular system; and expressing the gene in a bacterium or a yeast cell.

6. The method of claim 5, further comprising purifying the product from the step of expressing the CfIEM gene as a recombinant protein.

7. The method of claim 1, further comprising collecting the vanillin.

8. The method of claim 7, wherein the rate of conversion from isoeugenol to vanillin is higher than 80%.

9.-10. (canceled)

11. A method of producing vanillin using an isolated recombinant host cell comprising: (i) cultivating an isolated recombinant host cell in a medium; (ii) adding isoeugenol to the medium of (i) to begin the bioconversion of isoeugenol to vanillin; and (iii) extracting vanillin from the medium, wherein the isolated recombinant host cell has been transformed with a nucleic acid construct comprising a polynucleotide sequence encoding an isoeugenol monooxygenase, wherein the isoeugenol monooxygenase has an amino acid sequence with at least 70% identity to SEQ ID NO. 2.

12. (canceled)

13. The method of claim 11, wherein the isoeugenol monooxygenase has an amino acid sequence with at least 90% identity to SEQ ID NO. 2.

14. The method of claim 11, wherein the isoeugenol monooxygenase has an amino acid sequence with at least 95% identity to SEQ ID NO. 2.

15. A method of making a consumable product comprising the steps of: producing vanillin according to the method of claim 1; collecting the vanillin; and incorporating the vanillin into a consumable product.

16. The method of claim 15 comprising the step of admixing the vanillin with the consumable product.

17. The method of claim 15, wherein the vanillin is incorporated into the consumable product in an amount sufficient to impart a flavor note.

18. The method of claim 15, wherein the consumable product is selected from the group consisting of a flavored product, a food product, a food precursor product, an additive employed in the production of a foodstuff, a pharmaceutical composition, a dietary supplement, a nutraceutical product, and a cosmetic product.

19. The method of claim 15, wherein the vanillin is incorporated into the consumable product in an amount sufficient to impart a fragrance note.

20. The method of claim 15, wherein the consumable product is selected from the group consisting of a fragrant product, a cosmetic product, a toiletry product, and a house cleaning product.

21. An isolated recombinant host cell transformed with a nucleic acid construct comprising a polynucleotide sequence encoding an isoeugenol monooxygenase, wherein the isoeugenol monooxygenase has an amino acid sequence with at least 70% identity to SEQ ID NO. 2.

22. The isolated recombinant host cell of claim 21, wherein the polynucleotide sequence comprises a sequence that is at least 90% identical to the nucleic acid sequence of SEQ ID NO. 1.

23. The isolated recombinant host cell of claim 21 further comprising a vector containing the isolated nucleic acid sequence of SEQ ID NO. 5.

24. The isolated recombinant host cell of claim 21, wherein the host cell is selected from the group consisting of: a bacterium, a yeast, a filamentous fungus that is not Colletotrichum, a cyanobacterium, an alga, and a plant cell.

25. (canceled)


SEQUENCE LISTING (11 sequences)

SEQ ID NO: 1 - DNA, 1620 bp, Colletotrichum fioriniae
atggcaccgggtcgtaccgatgatgaagcagcagaagttgcaaccagcagcaaaacccatgcaattagcaattatggtctgaccaccaaatggcctgaagcatacgatctggcaggtagcaatctgccgtgtcgtctggaaggtgaaattggtgatctggttgttctgggtgaaattccgaaagaaattgatggcaccttttatcgtgttatgaccgatccgtttgttccgcctcatccgaataatgttccgctggatggtgatggtaatattagcgcatttcgcattaaagatggtcgcgtggatatgaaaatgcgttatgttgaaaccgagcgctataaactggaacgcaaagcaaataaagcactgtttggtctgtatcgcaatccgtttacacatcatccgtgtgttcgtgcagcagttgatagcaccgcaaataccaatgttgttctgtgggcagatcattttctggcactgaaagaaggtggtctgccgtatagcgttgatccgcagacactggaaaccatcaaatatgatccttttggccagatcaaagccaaaacctttaccgcacatccgaaaattgatccgtataccaatgaactggtggtgtttggttatgaagcacgtggtctggcaagcctggatattgttatttatgcactggatagcaacggcgtgaaacatgatgaacagtggattaaaagcccgtggtgtgcaccgattcatgattgtgcaattaccccgaattggattattctggccctgtggccgtttgaagcaagcgtggatcgtatgaaaaaaggcggtcatcattgggcatgggattatgatttaccggcaacctttattgttgcaccgcgtcgtagcagcaccaaactgcctccaggttggcagcctggtgaaagccgtgtttatagctggaaaaattgcatgctgattcataccggtggtgcatgggaaagcgaagatggtcagacactgtttatggaaaccacacgtgttcatgataatggctttccgttttttccgaccaatgaaggtgtttatccggcacctgatccgaaaagcgattatgttcgttggacatttgatctgagtcagccgaccaatagcaaagttcctgatccggaaattatcctggatatgcctgcagaatttccgcgtctggatgaacgttttctgagcaaaagctataattatgtgtggatcaacgtgtacatccacgaagatcagaacggtgatggtcataccattgttgaaggtctgaatggtctggccatgcataatagcaaaaccaaagaaaccaaatgctttaatgccggtgccgaaagtttttgtcaagaaccgatctttattccgcgtagtgatgatgcaccggaaggtgatggttgggttatgacaatggttgaacgtcgtggtgcaaatcgtaatgaaattgttgtgctggataccaaggatttcgaaaaaccgattgcagttattcagctgccgtttcatgttaaagcacaggttcatggtaattgggttgatcagcgtcgtctgaaagcatggcagaaactggcacgtgatgcagtgccggaaccgattagcggtcagggtgcactggaaccgctgtaa

SEQ ID NO: 2 - protein, 539 aa, Colletotrichum fioriniae
MAPGRTDDEAAEVATSSKTHAISNYGLTTKWPEAYDLAGSNLPCRLEGEIGDLVVLGEIPKEIDGTFYRVMTDPFVPPHPNNVPLDGDGNISAFRIKDGRVDMKMRYVETERYKLERKANKALFGLYRNPFTHHPCVRAAVDSTANTNVVLWADHFLALKEGGLPYSVDPQTLETIKYDPFGQIKAKTFTAHPKIDPYTNELVVFGYEARGLASLDIVIYALDSNGVKHDEQWIKSPWCAPIHDCAITPNWIILALWPFEASVDRMKKGGHHWAWDYDLPATFIVAPRRSSTKLPPGWQPGESRVYSWKNCMLIHTGGAWESEDGQTLFMETTRVHDNGFPFFPTNEGVYPAPDPKSDYVRWTFDLSQPTNSKVPDPEIILDMPAEFPRLDERFLSKSYNYVWINVYIHEDQNGDGHTIVEGLNGLAMHNSKTKETKCFNAGAESFCQEPIFIPRSDDAPEGDGWVMTMVERRGANRNEIVVLDTKDFEKPIAVIQLPFHVKAQVHGNWVDQRRLKAWQKLARDAVPEPISGQGALEPL

SEQ ID NO: 3 - DNA, 1437 bp, Pseudomonas putida
atggcgagactcaaccgcaacgacccgcaattagtaggaacacttctccccacccgtatcgaggcagacttgttcgatctagaggttgacggcgaaatcccaaaatcaataaatggaacgttctaccgtaatacgccagaacctcaagttaccccgcaaaaattccacaccttcatagatggagatggaatggcctctgccttccacttcgaagatggtcatgtcgacttcatcagtcgctgggttaaaaccgctcgattcacggccgaacgactagcgcgaaaatcgctatttggcatgtacagaaacccctataccgacgacaccagtgtaaaaggactagaccgcaccgttgccaatacaagcatcattagccatcacggcaaggtgctggcggtgaaggaagacggcctaccgtacgaactggatcctcgtacacttgaaactcgcggacgcttcgactacgacggccaagttaccagccaaacccacaccgcccatccaaaatatgacccggaaacgggtgacttgttgttcttcggttcggcagctaagggcgaagcaactccagacatggcctattacattgtcgacaagcacggcaaggtgacacatgaaacttggtttgagcagccctatggcgcattcatgcacgactttgccattacccgaaattggtccattttcccaattatgccggccaccaacagcctgtcccgcctcaaggcgaaacagccaatttatatgtgggagccggaactgggcagctacattggcgtactgccgcgccgcggccagggcagtcagattcgctggctcaaggcaccggcgctctgggtatttcatgttgtgaatgcttgggaagtcggaaccaagatttatatcgaccttatggaaagtgaaatcctgccgttccccttccccaactcacaaaaccaacccttcgcccctgagaaagccgtaccacgcctgactcgttgggaaattgacctcgatagcagcagcgacgagatcaagcgaacccggctacacgatttctttgcggaaatgccaatcatggattttcgcttcgccctgcaatgcaaccgctatggctttatgggggtggacgatccacgcaaaccacttgcgcatcagcaggccgagaagatatttgcgtacaactcactcggcatctgggacaaccaccgaggtgactacgacctctggtactccggagaagcctcggcggcccaggagccggccttcgtccctagaagtccgaccgccgccgaaggtgatgggtacttgctgaccgtggttggtcgtctcgatgaaaatcgcagtgatctggtaattctcgacactcaagacatccagtctggtcccgtggcaaccatcaagctgccattccggttaagggccgctctccatggctgctgggtacccagaccttaa

SEQ ID NO: 4 - protein, 478 aa, Pseudomonas putida [sequence omitted]

SEQ ID NO: 5 - DNA, 2325 bp, Artificial Sequence
  Plasmid
ggcagtgagcgcaacgcaattaatgtgagttagctcactcattaggcaccatcgtttaggcaccccaggctttacactttatgcttccggctcgtataatgtgtggaattgtgagcggataacaatttcaactataagaaggagatatacatatggcggatccgaattcggcgcgccagatctcaattggatatcggccggccgacgtcggtaccgcggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaaaatcaaaggatcttcttgagatcctttttttctgcgcgtaatctgctgcttgcaaacaaaaaaaccaccgctaccagcggtggtttgtttgccggatcaagagctaccaactctttttccgaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcatcgatttttgtgatgctcgtcaggggggcggagcctatggaaaaacgccagcaacgcagaaaggcccacccgaaggtgagccaggtgattacatttgggccctcatcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcatttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatagctcctgaaaatctcgataactcaaaaaatacgcccggtagtgatcttatttcattatggtgaaagttggaacctcttacgtgccgatcaagtcaaaagcctccggtcggaggcttttgactttctgctatggaggtcaggtatgatttaaatggtcagtattgagcgatatctagagaattcgtc

SEQ ID NO: 6 - DNA, 39 bp, Artificial Sequence
  Primer
ggaattccatatggcaccgggtcgtaccgatgatgaagc

SEQ ID NO: 7 - DNA, 42 bp, Artificial Sequence
  Primer
aaggaaaaaagcggccgccagcggttccagtgcaccctgacc

SEQ ID NO: 8 - DNA, 45 bp, Artificial Sequence
  Primer
aaggaaaaaagcggccgcttacagcggttccagtgcaccctgacc

SEQ ID NO: 9 - DNA, 36 bp, Artificial Sequence
  Primer
ggaattccatatgagcgcaccggaagaacatcatgg

SEQ ID NO: 10 - DNA, 42 bp, Artificial Sequence
  Primer
aaggaaaaaagcggccgctgccagcggttccagtgcaccacg

SEQ ID NO: 11 - DNA, 45 bp, Artificial Sequence
  Primer
aaggaaaaaagcggccgcttatgccagcggttccagtgcaccacg